(19) United States
(12) Patent Application Publication
(10) Pub. No.: US 2005/0177045 A1
(43) Pub. Date: Aug. 11, 2005

(54) cMUT devices and fabrication methods

(75) Inventors: F. Levent Degertekin, Decatur, GA (US); Jeffrey John McLean, Baton Rouge, LA (US); Joshua Glenn Knight, Columbus, IN (US)

Correspondence Address: TROUTMAN SANDERS LLP, BANK OF AMERICA PLAZA, SUITE 5200, 600 PEACHTREE STREET , NE, ATLANTA, GA, 30308-2216, US

(73) Assignee: Georgia Tech Research Corporation, Atlanta, GA (US)

(21) Appl. No.: 11/053,672

(22) Filed: Feb. 7, 2005

Related U.S. Application Data
(60) Provisional application No. 60/542,378, filed on Feb. 6, 2004.

(51) Int. Cl. H01L 21/00; A61B 8/02; A61B 8/12

(52) U.S. Cl. 600/457; 600/447; 367/140; 327/100

(57) ABSTRACT

Fabrication methods for capacitive-micromachined ultrasound transducers (“cMUT”) and cMUT imaging array systems are provided. cMUT devices fabricated from low process temperatures are also provided. In an exemplary embodiment, a process temperature can be less than approximately 300 degrees Celsius. A cMUT fabrication method generally comprises depositing and patterning materials on a substrate (400). The substrate (400) can be silicon, transparent, other materials. In an exemplary embodiment, multiple metal layers (405, 410, 415) can be deposited and patterned onto the substrate (400); several membrane layers (420, 435, 445) can be deposited over the multiple metal layers (405, 410, 415); and additional metal layers (425, 430) can be disposed within the several membrane layers (420, 435, 445). The second metal layer (410) is preferably resistant to etchants used to etch the third metal layer (415) when forming a cavity (447). Other embodiments are also claimed and described.

### CROSS REFERENCE TO RELATED APPLICATION AND PRIORTY CLAIM

This Application claims the benefit of U.S. Provisional Application Ser. No. 60/542,378 filed on 6 Feb. 2004.

### TECHNICAL FIELD

The invention relates generally to chip fabrication, and more particularly, to fabricating capacitive micromachined ultrasonic transducers and capacitive micromachined ultrasonic transducer imaging arrays.

### BACKGROUND

Capacitive micromachined ultrasonic transducer (“cMUT”) devices generally combine mechanical and electronic components in very small packages. Typically, the mechanical and electronic components operate together. Because cMUTs are typically very small and have both mechanical and electrical parts, they are commonly referred to as micro-electronic mechanical systems (“MEMS”) devices.

MEMS manufacturing processes have launched many innovations in many different technical fields. The medical device field has greatly benefited from MEMS technology. MEMS technology enables medical device manufacturing from devices such as cMUTs and cMUT imaging arrays. cMUT technology, by virtue of its miniscule nature, enables medical professionals to obtain critical medical information from within a patient's body while utilizing minimally invasive medical procedures. Intravascular imaging and interventions is a particular area where miniaturized devices are critical. To ensure that cMUT devices can function correctly in imaging applications, device manufacturers have devised fabrication approaches and techniques to improve cMUT imaging technology.

Conventional cMUT fabrication processes make use of low-pressure chemical vapor depositions (“LPCVD”) for cMUT membrane formation and sealing. The high process temperatures (approximately 900 degrees Celsius) of LPCVD make post-process complimentary metal oxide semiconductor (“CMOS”) integration impossible. These high temperatures also eliminate the possibility of using transparent substrates, thereby eliminating optical detection methods.

Post-process fabrication generally includes fabricating cMUTs on substrates fabricated with electronic devices, such as CMOS transistors. Some known post-process integration approaches use vias that enable cMUTs to be flip-chip bonded to a signal-processing chip as a means for hybrid integration. Yet, such approaches do not allow a CMOS device to be fabricated into a cMUT device, but rather merely bond a CMOS device to a cMUT after fabrication. A major drawback to this approach is the complicated fabrication process.

Other currently utilized manufacturing techniques have similar drawbacks. A recently developed technique for cMUT post-process integration utilizes plasma enhanced chemical vapor deposition (“PECVD”). This technique, however, both generates a large cMUT cavity, approximately 4000 Angstroms or larger, and requires high DC bias voltages. Additionally, the PECVD process temperature (approximately 400-500 degrees Celsius) is still too high for CMOS integration as it can destroy CMOS electronics. Similarly, wafer bonding techniques used to improve cMUT membrane uniformity in cMUT fabrication requires high bonding temperatures, thus making post-process CMOS integration impossible.

An additional approach to cMUT electronics integration involves post-processing cMUTs directly over CMOS electronics. This process makes use of polymer sacrificial layers under a silicon nitride membrane formed with PECVD, but generates gaps of approximately 1-2 micrometers in the membrane. To operate cMUTs at high frequencies, the membranes must be small and stiff to achieve typical desired resonant frequencies. Due to the gaps in this process, the resulting membrane is not suitable for efficient cMUT operation at high frequencies. Stiff membranes coupled with large gaps can require prohibitively high collapse voltages for efficient cMUT operation.

Parasitic capacitance is another disadvantage of conventional cMUT devices and fabrication processes. Parasitic capacitance can arise from cMUT electrical interconnections and connections to associated amplifying electronics. If not limited adequately, parasitic capacitance can cause a cMUT device to function improperly, thus limiting its ability to provide quality images or data.

Therefore, there is a need in the art for a cMUT fabrication method that enables electronic integration via post-CMOS processing without sacrificing cMUT device performance.

Additionally, there is a need in the art for fabricating cMUTs having reduced parasitic capacitances and utilizing optical displacement detection methods.

Additionally, there is a need in the art for a less complex, yet effective, cMUT manufacturing process.

It is to the provision to such cMUT fabrication and cMUT imaging array fabrication that the present invention is primarily directed.

### BRIEF SUMMARY OF THE INVENTION

The present invention comprises cMUT array transducer fabrication methods and systems. The present invention provides cMUTs for imaging applications that can be fabricated directly on top of CMOS electronics, which can be especially useful in medical imaging applications. The cMUTs can be fabricated on dielectric or transparent substrates, such as, but not limited to, quartz or sapphire, to reduce device parasitic capacitance, thus improving electrical performance and enabling optical detection methods to be used. Additionally, cMUTs produced according to the present invention may be used in immersion applications such as intravascular catheters and ultrasound imaging.

The present cMUT device can comprise a cMUT coupled to a substrate and a circuit proximate the cMUT adapted to receive and direct at least one of an optical and electrical signal to and from the cMUT. The substrate can be a silicon substrate. Additionally, a circuit can be embedded in the substrate proximate the cMUT to receive and direct electronic signals to and from the cMUT. The present cMUT can comprise electrode materials and sacrificial layer materials that are selected such that an etchant used to etch the sacrificial layer will not etch the electrode, wherein an isolation layer is not needed between the electrode and the sacrificial layer.

A transparent substrate can be used and a circuit embedded in the transparent substrate proximate the cMUT to receive and direct optical signals to and from the cMUT. In other preferred embodiments, a combination of a transparent substrate with a silicon layer, such as silicon-on-sapphire wafers, can be used and a circuit embedded in the silicon layer on the transparent substrate proximate the cMUT to receive and direct optical signals to and from the cMUT. The surface of the transparent substrate on which the cMUTs are built can incorporate a stack of thin dielectric layers to increase reflectivity in a particular optical wavelength range.

The present cMUT device fabrication process comprises depositing and patterning layers of materials on a substrate. For example, a preferred cMUT fabrication process includes depositing and patterning a first conductive layer on a substrate; depositing and patterning a sacrificial layer on the first conductive layer; depositing and patterning a first membrane layer on the sacrificial layer; depositing and patterning a second conductive layer on the first membrane layer; depositing and patterning a second membrane layer on the second conductive layer; and etching the sacrificial layer. The process temperatures utilized are preferably less than approximately 300 degrees Celsius, and more preferably less than approximately 250 degrees Celsius. The layers of materials can comprise Chromium, Gold, Aluminum, and/or silicon nitride.

These and other features as well as advantages, which characterize the various preferred embodiments of present invention, will be apparent from a reading of the following detailed description and a review of the associated drawings.

### BRIEF DESCRIPTION OF THE DRAWINGS

FIG. 1 is an illustration of a cross-sectional view of a cMUT fabricated on a substrate in accordance with a preferred embodiment of the present invention.

FIG. 2 is an illustration of a cross-sectional view of a cMUT fabricated on a substrate in accordance with another preferred embodiment of the present invention.

FIG. 3 is an illustration of a fabrication process utilized to produce a cMUT on a substrate in accordance with a preferred embodiment of the present invention.

FIGS. 4A and 4B (collectively FIG. 4) are an illustration of another fabrication process utilized to produce a cMUT on a substrate in accordance with another preferred embodiment of the present invention.

FIG. 5 is a logic flow diagram depicting a method to fabricate a cMUT device on a substrate in accordance with a preferred embodiment of the present invention.

FIG. 6 is an illustration of a cMUT imaging array system formed in a ring-annular array on a substrate in accordance with a preferred embodiment of the present invention.

FIG. 7 is an illustration of a cMUT imaging array system formed in a side-looking array on a substrate in accordance with a preferred embodiment of the present invention.

### DETAILED DESCRIPTION OF PREFERRED EMBODIMENTS

cMUTs have been developed as an alternative to piezoelectric ultrasonic transducers particularly for micro-scale and array applications. Since cMUTs are surface micromachined, they can be fabricated into one or two-dimensional arrays and customized for specific applications, and can have performance comparable to piezoelectric transducers in terms of bandwidth and dynamic range. A cMUT device typically incorporates a membrane, with an electrode, suspended above a conductive substrate or another electrode coupled to a substrate. The membrane can have elastic properties enabling it to fluctuate in response to stimuli. For example, stimuli may include, but are not limited to, external forces exerting pressure on the membrane and electrostatic forces applied through cMUT electrodes. cMUTs can transmit and receive acoustical waves. To transmit an acoustic wave, an AC signal and a large DC bias voltage are applied to the membrane. The DC voltage pulls down the membrane where the transduction is efficient and linearizes the cMUT device response. The AC voltage sets the membrane into motion at a desired frequency and generates an acoustic wave in a surrounding fluid. To receive an acoustic wave, the capacitance change is measured when an impinging acoustic wave sets the membrane into motion. If cMUT imaging array elements have a small, mechanically active area covered with an electrode, the capacitance change will also be small, and thus can easily be overwhelmed by parasitic capacitance. Accordingly, it is typically desirable to counteract the causes of such parasitic capacitance.

Parasitic capacitance is typically found in two different areas in connection with cMUTs—each requiring a unique solution. A first source of parasitic capacitance is the area where bond pads and metal traces on a substrate overlap a bottom electrode. Since standard cMUT processes make use of a doped silicon bottom electrode, parasitic capacitance can dominate the active capacitance of the device. A patterned metal bottom electrode can be used to reduce this on-chip capacitance. For a silicon substrate, this patterned electrode can be formed on a dielectric layer deposited over the silicon substrate. The dielectric layer can be silicon oxide, silicon nitride or a similar thin film dielectric layer. The use of the metal bottom electrode also enables cMUT fabrication on dielectric substrates, such as quartz. With a transparent substrate, optical detection schemes, which are independent of device capacitance, can be implemented to improve the cMUT performance. Indeed, the bottom electrode of the cMUT can be patterned in the form of a diffraction grating. Although materials such as doped polysilicon or amorphous silicon can also be used for the bottom electrode, metals have higher electrical conductivity and optical reflectivity, which are desirable for optical detection.

A second source of parasitic capacitance comes from electrical interconnects to amplifying electronics. This source of parasitic capacitance can be reduced through hybrid or monolithic integration with the electronics which is typically implemented using CMOS technology.

The present invention provides CMOS compatible cMUT fabrication processes without performance tradeoffs and a reduced number of process steps, as compared to hybrid integration using through wafer vias. Exemplary equipment for fabricating cMUTs according to the present invention can include, but are not limited to, a PECVD system, a dry etching system, a metal sputtering system, a wet bench, and photolithography equipment. The present invention can utilize low-temperature PECVD processes for the deposition of the low stress silicon nitride structural layer at approximately 250 degrees Celsius, which is preferably the maximum process temperature when a metal sacrificial layer is used. Alternatively, the present invention according to other preferred embodiments can utilize an amorphous silicon sacrificial layer deposited as a sacrificial layer at approximately 300 degrees Celsius.

The process temperatures of various embodiments of the present invention enable post-process CMOS electronics integration without compromising cMUT performance. Post-process CMOS integration typically involves fabricating a device on a substrate containing electronics, such as CMOS-type transistor devices. When the cMUTs are fabricated on a substrate containing electronics, additional processing steps may be desirable before fabricating the cMUTs. For example, these steps can include depositing a dielectric layer at a temperature below approximately 400 degrees Celsius over the CMOS electronics, opening vias in the dielectric layer to provide conductive paths to the desired nodes of the CMOS electronics, and depositing a conductive layer to fill the vias. The conductive material can be patterned using photolithographic techniques. A final step in preparing a substrate with CMOS electronics can include polishing the substrate surface to smooth the surface. After this step, the cMUTs are electrically isolated from the CMOS electronics except for the via locations. Thus, at least one of the cMUT electrodes can directly contact the CMOS electronics with reduced parasitic capacitance. The polishing step is generally desired so that a cMUTs can be fabricated on a smooth surface, preferably a surface with less than approximately 10 nm rms (“root mean square”) surface roughness.

The electrode size and location can be changed to reduce parasitic capacitance and optimize device performance since a dielectric membrane can be used. Those of ordinary skill in the art will be familiar with various methods for reducing parasitic capacitance and optimizing device performance. The cMUT membranes can be sealed using PECVD silicon nitride, thus allowing for immersion operation and eliminating the need for long sealing channels typically required by LPCVD silicon nitride sealing. Additionally, preferred embodiments of the present invention enable cMUT fabrication on optically transparent dielectric substrates using a patterned metal bottom electrode, reducing parasitic capacitance and providing an opportunity for optical detection.

Transparent substrates can include, but are not limited to, quartz and silicon type substrates. The present processes can be low-temperature fabrication processes capable of producing interdigital cMUTs for microfluidic applications and ring-annular cMUT imaging arrays for forward looking intravascular ultrasound imaging (“IVUS”) applications.

Referring now the drawings, in which like numerals represent like elements, and like shading represents like components or materials, preferred embodiments of the present invention are herein described.

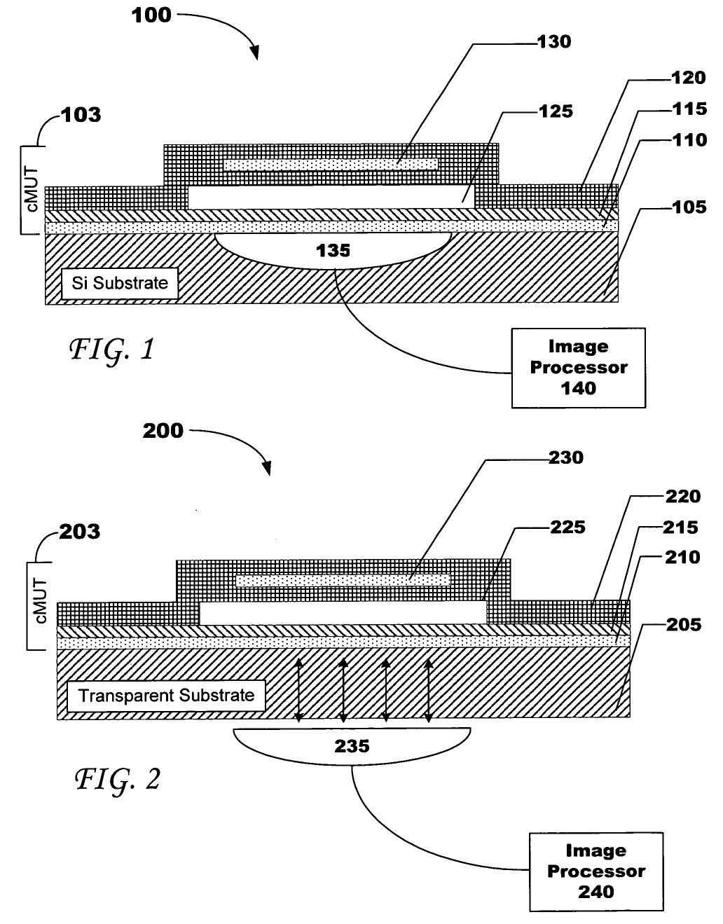

FIG. 1 is an illustration of a cross-sectional view of a cMUT fabricated on a substrate in accordance with a preferred embodiment of the present invention. The cMUT device 100 generally includes a cMUT 103 in combination with a substrate 105. An exemplary cMUT 103 comprises a bottom electrode 110, an isolation layer 115, a membrane layer 120, a cavity 125, and a top electrode 130. The isolation layer 115 may not be used in some embodiments so the bottom electrode 110 can be exposed to the cavity 125.

The device 100 can further comprise an integrated electronic circuit 135 coupled to the cMUT 103 adapted to receive and provide electronic signals to and from the cMUT 103 through the bottom electrode 110 and top electrode 130. As shown, a portion of the membrane layer 120 is suspended above the cavity 125, and the top electrode 130 is disposed within the membrane layer 120.

The distance between the two electrodes 110, 130 can fluctuate. The top electrode 130 can move or fluctuate relative to the bottom electrode 110, as the membrane layer 120 can be adapted to fluctuate when an external pressure is applied to the membrane layer 120, or when an adequate voltage is applied to the electrodes 110, 130.

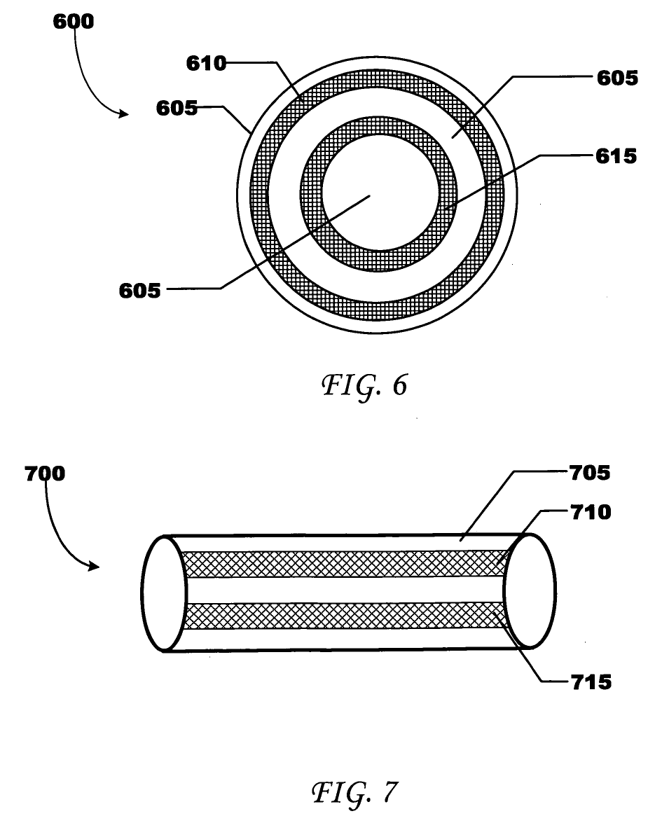

A plurality of devices 100 can be used to form a cMUT imaging array as discussed in greater detail with reference to FIGS. 6-7. For example, a ring-annular cMUT imaging array can be formed on an outer periphery of the substrate 105. A ring-annular array can include various types of annular ring arrays or annular arrays. In other exemplary embodiments, the device 100 can be arranged in different topologies or arrangements. For example, a plurality of devices 100 can be arranged in a side looking arrangement, or the substrate can be placed at an angle to a central axis of a catheter to produce images at a particular viewing angle. In other preferred embodiments, the cMUT imaging array can be arranged in an annular array with multiple rings, or a sparse or fully populated linear 1-D or 2-D array. Additionally, a plurality of devices 100 can be formed on the same substrate using an exemplary embodiment of the present invention.

The substrate 105 can be made with various materials, including, but not limited to, opaque or transparent materials such as silicon, quartz, glass, fused silica, or sapphire. Those skilled in the art will recognize that transparent materials can include substrates that are optically transparent to a predetermined wavelength of light directed at the substrate. If the substrate 105 is silicon, the substrate 105 can be doped, and can be adapted to enable an electronic or optical signal to pass through the silicon substrate. A silicon substrate can contain integrated electronics or optical circuits to generate and process input and output signals for the device 100. A transparent substrate can be adapted to enable an optical signal to pass through the transparent substrate. For example, a silicon substrate can be used as a transparent substrate when using light of a predetermined wavelength as an optical signal. In some embodiments, the substrate 105 has a thickness in the range of approximately 10 micrometers to approximately 1 millimeter.

The device 100 can be utilized to sense images. For example, the device 100 can be adapted to utilize a fluctuating capacitance in response to environmental factors (such as external applied pressures), and to provide the fluctuating capacitance to a system that produces an image from the measured capacitance. An integrated electronic circuit 135 can sense electronic signals produced by the bottom electrode 110 and the top electrode 130, and provide those electronic signals to an image processor 140. The electrodes 110, 130 can be coupled to an integrated electronic circuit 135 through vias (not shown) formed in various layers of the device 100. The integrated electronic circuit can comprise CMOS electronic devices or other transistor-type devices. Those skilled in the art will be familiar with various methods for translating capacitance measurements on a cMUT imaging array into an image using an image processor 140 or similar system.

Additionally, the device 100 can be utilized to sense a variety of real-time information. For example, the device can be adapted to be a pressure sensor, temperature sensor, flow sensor, a Doppler flow sensor, an electrical resistivity sensor, a fluid viscosity sensor, a gas sensor, a chemical sensor, an accelerometer, or other desirable sensors. In addition when used in imaging applications, the device 100 can be a florescence or optical reflectivity sensor adapted to measure reflected and scattered light from surrounding tissue and fluids to monitor optical parameters such as reflectivity and fluorescence.

The device 100 can be fabricated from a plurality of layers. Conductive materials can form conductive layers, which can be patterned to form the electrodes 110, 130. For example, the conductive material can be a doped silicon surface of the substrate 105, a doped polysilicon layer, a conductive metal, or other suitable conductive materials. The electrodes 110, 130 can be coupled to signal generation and detection circuits such as the integrated electronic circuit 135 embedded in the silicon substrate 105. In some embodiments, the signal generation and detection circuits are embedded within the substrate 105 and can be located on another chip proximate the substrate 105.

A challenge in using embedded integrated electronic circuitry is that the integrated electronic parts can be damaged if subjected to high temperatures utilized during device fabrication. In an exemplary embodiment of the present invention, fabrication of a cMUT above embedded integrated electronics takes place at a relatively low temperature, thereby avoiding the use of damaging heat levels.

In yet another embodiment of the present invention, a cMUT device is fabricated using transparent substrates adapted to reflect light to provide current status information. For example, a cMUT device can have electrodes coated with a reflective material, or can be made from a material having natural reflective properties. Additionally, a bottom electrode used with optical detection methods and a transparent substrate can be patterned into a diffraction grating. For cMUTs fabricated on transparent substrates, some of the electrical connections can be made using a transparent metal layer, such as indium tin oxide. A transparent substrate according to some embodiments of the present invention is formed from materials such as, but not limited to, glass, quartz, tin-oxide, or fused silica using a low temperature fabrication process. Other transparent substrates can be formed from materials such as sapphire.

FIG. 2 is an illustration of a cross-sectional view of a cMUT device fabricated on a substrate fabricated in accordance with another embodiment of the present invention. The cMUT device 200 generally includes a cMUT 203 in combination with a transparent substrate 205. The substrate 205 can be, but is not limited to, glass, quartz, or sapphire. In cases where silicon is substantially transparent at the wavelength of a particular light source, silicon can also be used as a transparent substrate.

The cMUT 203 generally comprises a bottom electrode 210, an isolation layer 215, a membrane layer 220, a cavity 225, and a top electrode 230. The isolation layer 215 may not be used in some embodiments. As shown, a portion of the membrane layer 220 is suspended above the cavity 225, and the top electrode 230 is embedded within the membrane layer 220. The device 200 can also include an optical detection circuit 235 adapted to receive and provide optical signals to and from the cMUT 203.

The optical detection circuit 235 can be adapted to optically interrogate the cMUT 203. For example, optical detection circuit 235 can be adapted to direct or provide an optical beam to the cMUT 203 and to receive a reflected optical beam from the cMUT 203. The arrows shown within the transparent substrate 205 in FIG. 2 illustrate that optical signals can pass through the transparent substrate 205, thus optically coupling the cMUT 203 and the optical detection circuit 235. The optical detection circuit 235 can be adapted to determine the current status of the cMUT 203 by measuring the intensity of a reflected optical beam. Current status information can reveal the capacitance associated with a cMUT at various time intervals. One exemplary method of analyzing the reflected light beam includes comparing the intensity of the reflected light beam to the intensity of the light beam directed to the cMUT 203. The optical detection circuit 235 can communicate with an image processor 240 capable of producing an image from the information sensed by the optical detection circuit 235. The optical detection circuit 235 can be fabricated on a separate substrate or on the same substrate as cMUT 203. For example, a separate substrate can be bonded to the transparent substrate 205 so that the detection circuit 235 is located proximate the cMUT 203.

Using transparent substrates in cMUT fabrication according to the present invention provides several advantages. One advantage associated with transparent substrates is the ease of manufacturing the device, because electrical connections are typically not necessary since optic signals are utilized. Another advantage is that optical interrogation uses light signals, not electronic signals that produce electromagnetic radiation. Thus, optical interrogation may alleviate crosstalk problems associated with electromagnetic radiation. An additional advantage is that transparent substrates provide cMUT devices with little to no parasitic capacitance.

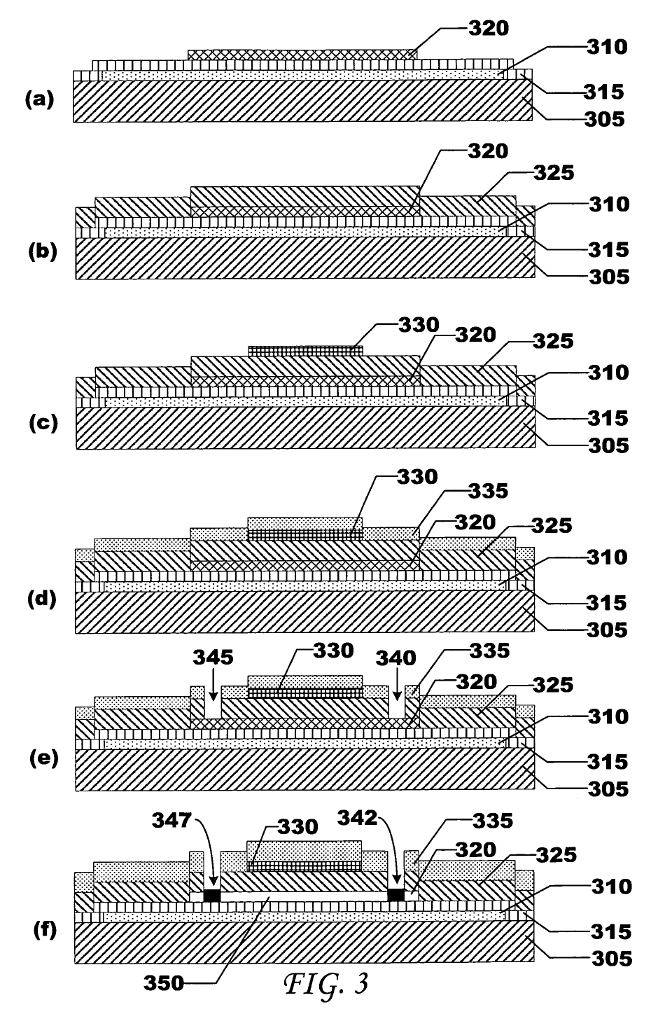

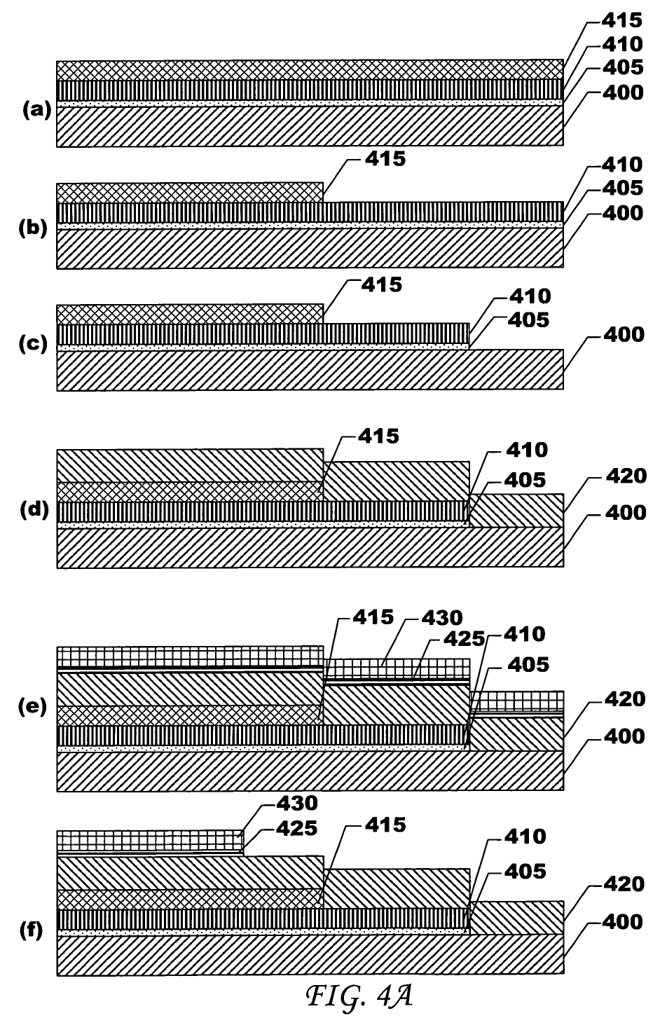

FIG. 3 is an illustration of a fabrication process utilized to produce a cMUT on a substrate. Typically, the fabrication process is a build-up process that involves depositing various layers of materials on a substrate, and patterning the various layers in predetermined configurations to fabricate the cMUT on the substrate.

In a preferred embodiment of the present invention, a photoresist such as Shipley S-1813 is used to lithographically define various layers of a cMUT. Such a photoresist material does not require the use of the conventional high temperatures for patterning vias and material layers. Alternatively, other materials may be used.

The first step in the present fabrication process provides a bottom electrode 310 on a substrate 305. In some embodiments, the substrate 305 contains integrated electronics. Alternatively, a second substrate located proximate the substrate 305 containing suitable detection electronics can be used. A conductive material, such as conductive metals, can form the bottom electrode 310. The bottom electrode 310 can be formed by doping a silicon substrate 305 or by depositing and patterning a conductive material layer (such as metal) on the substrate 305. Yet, with a doped silicon bottom electrode 310, all non-moving parts of a top electrode can increase parasitic capacitance, thus degrading device performance and prohibiting optical detection techniques for most of the optical spectrum.

To overcome these disadvantages, a patterned bottom electrode 310 can be used. As shown in FIG. 3a, the bottom electrode 310 can be patterned to have a different length than the substrate 305. By patterning the bottom electrode 310, device parasitic capacitance can be significantly reduced. Also, the bottom electrode 310 enables cMUTs to be fabricated on dielectric substrates, such as quartz. Low process temperature can be advantageous when post-processing cMUTs over integrated electronics such as CMOS circuitry. Aluminum, chromium, and gold are exemplary metals that can be used to form the bottom electrode 310. In one preferred embodiment of the present invention, the bottom electrode 310 has a thickness of approximately 1500 Angstroms, and after deposition, can be patterned as a diffraction grading, or to have various lengths. In another exemplary embodiment, the bottom electrode 310 comprises Aluminum having a thickness of approximately 1200 Angstroms and Chromium having a thickness of approximately 300 Angstroms.

In a next step, an isolation layer 315 is deposited. The isolation layer 315 can isolate the bottom electrode 310 from other layers placed on the bottom electrode 310. The isolation layer 315 can be silicon nitride, and preferably has a thickness of approximately 1500 Angstroms. For example, a Unaxis 790 PECVD system can be used to deposit the isolation layer 315 at approximately 250 degrees Celsius. The isolation layer 315 protects the bottom electrode 310 or the substrate 305 from etchants used during the cMUT fabrication process. Once deposited onto the bottom electrode layer 310, the isolation layer 315 can be patterned to a predetermined thickness.

In an alternative preferred embodiment, an isolation layer 315 is not utilized. Rather than using an isolation layer 315, the bottom electrode is made using a material that is not affected by an etchant used to etch the sacrificial layer 320, thus being resistant to the etchant that removes the sacrificial layer 320.

After the isolation layer 315 is deposited, a sacrificial layer 320 is deposited onto the isolation layer 315. The sacrificial layer 320 is preferably only a temporary layer, and is etched away. When an isolation layer 315 is not used, the sacrificial layer 320 can be deposited directly on the bottom electrode 310. The sacrificial layer 320 is used to hold a space while additional layers are deposited during the process. The sacrificial layer 320 can be used to help create a hollow chamber such as a cavity or a via. The sacrificial layer 320 can be formed with amorphous silicon that can be deposited using a Unaxis 790 PECVD system at approximately 300 degrees Celsius and patterned with a reactive ion etch (“RIE”). Sputtered metal can also be used to form the sacrificial layer 320. The sacrificial layer 320 can be patterned into different sections, various lengths, and different thicknesses to provide varying geometrical configurations for a resulting cavity or via.

A first membrane layer 325 is then deposited onto the sacrificial layer 320, as shown in FIG. 3b. For example, the first membrane layer 325 can be deposited using a Unaxis 790 PECVD system. The first membrane layer 325 can be a layer of silicon nitride or amorphous silicon, and can be patterned to have a thickness of approximately 6000 Angstroms. The thickness of the first membrane layer 325 can vary depending on the particular implementation. Depositing the first membrane layer 325 over the sacrificial layer forms a vibrating membrane of the cMUT.

After patterning the first membrane layer 325, a second conductive layer 330 can be deposited onto the first membrane layer 325 as illustrated in FIG. 3(c). The second conductive layer 330 can form the top electrode of a cMUT. The second conductive layer 330 is generally formed from metals such as Aluminum, Chromium, or combinations thereof. In an exemplary embodiment, the second conductive layer comprises Aluminum having a thickness of approximately 1200 Angstroms and Chromium having a thickness of approximately 300 Angstroms. Aluminum provides good electrical conductivity, and Chromium protects the Aluminum from oxidation. In other embodiments, other metals, such as Gold, can be utilized as the second conductive layer 330. Additionally, the second conductive layer 330 can be the same conductive material or a different conductive material than the first conductive layer 310.

In a next step, a second membrane layer 335 is deposited over the second conductive layer 330 as illustrated in FIG. 3(d). The second membrane layer 335 increases the thickness of the cMUT membrane at this point in fabrication (formed by the first and second membrane layers 325, 335), and can serve to protect the second conductive layer 330 from etchants used during cMUT fabrication. The second membrane layer can be approximately 6000 Angstroms thick. In some embodiments, the second membrane layer 335 is adjusted using deposition and patterning techniques so that the second membrane layer 335 has an optimal geometrical configuration. Preferably, once the second membrane layer 335 is adjusted according to a predetermined geometric configuration, the sacrificial layer 320 is etched away, leaving a cavity 350.

To enable etchants to reach the sacrificial layer 320, apertures 340, 345 can be etched through the first and second membrane layers 325, 335 using a RIE process. As shown in FIG. 3(e), access to the sacrificial layer 320 is formed at apertures 340, 345 by etching away the first and second membrane layers 325, 335. When an amorphous silicon sacrificial layer 320 is used, one must be aware of the selectivity of the etch process to silicon. If the etching process has low selectivity, one can easily etch through the sacrificial layer 320, the isolation layer 315, and down to the substrate 305. If this occurs, the etchant used for release can attack the substrate 305 and can destroy a cMUT device. When the bottom electrode 310 is formed from a metal that is resistant to the etchant used with the sacrificial layer, the metal layer can act as an etch stop and protect the substrate 305. Those skilled in the art will be familiar with various etchants and matching the etchants to the materials being etched. After the sacrificial layer 320 is etched, the cavity 350 can be sealed with seals 342, 347, as shown in FIG. 3f.

The cavity 350 can be formed between the isolation layer 315 and the membrane layers 325, 335. The cavity 350 can also be disposed between the bottom conductive layer 310 and the first membrane layer 325. The cavity 350 can be formed to have a predetermined height in accordance with exemplary embodiments of the present invention. The cavity 350 enables the cMUT membrane formed by the first and second membrane layers 325, 335 to fluctuate and resonate in response to stimuli. After the cavity 350 is formed by etching the sacrificial layer 320, the cavity 350 can be vacuum sealed by depositing a sealing layer (not shown) on the second membrane layer 335. Those skilled in the art will be familiar with various methods for setting a pressure in the cavity 350 and then sealing it to form a vacuum seal.

The sealing layer is typically a layer of silicon nitride, having a thickness greater than the height of the cavity 350. In an exemplary embodiment, the sealing layer has a thickness of approximately 4500 Angstroms, and the height of the cavity 350 is approximately 1500 Angstroms. In alternative embodiments, the second membrane layer 335 is sealed using a local sealing technique or sealed under predetermined pressurized conditions. Sealing the second membrane layer 335 can adapt the cMUT for immersion applications. After depositing the sealing layer, the thickness of the composite cMUT membrane can be adjusted by etching back the sealing layer since the cMUT membrane may be too thick to resonate at a desired frequency. A dry etching process, such as RIE, can be used to etch the sealing layer.

A final step in the present cMUT fabrication process prepares the cMUT for electrical connectivity. Specifically, RIE etching can be used to etch through the isolation layer 315 on the bottom electrode 310, and the second membrane layer 335 on the top electrode 330, making the electrodes 310, 330 accessible.

Additional bond pads may be formed and connected to the electrode. Bond pads enable external electrical connections to be made to the top and bottom electrodes 310, 330 with wire bonding. In some embodiments, gold can be deposited and patterned on the bond pads to improve the reliability of the wire bonds.

In an alternative embodiment of the present invention, the sacrificial layer 320 can be etched after depositing the first membrane layer 325. This alternative embodiment invests little time in the cMUT device before performing the step of etching the sacrificial layer 320 and releasing the membrane formed by the membrane layers. Since the top electrode 330 has not been deposited, there is no risk that pinholes in the second membrane layer 335 could allow the top electrode 330 to be destroyed by etchants.

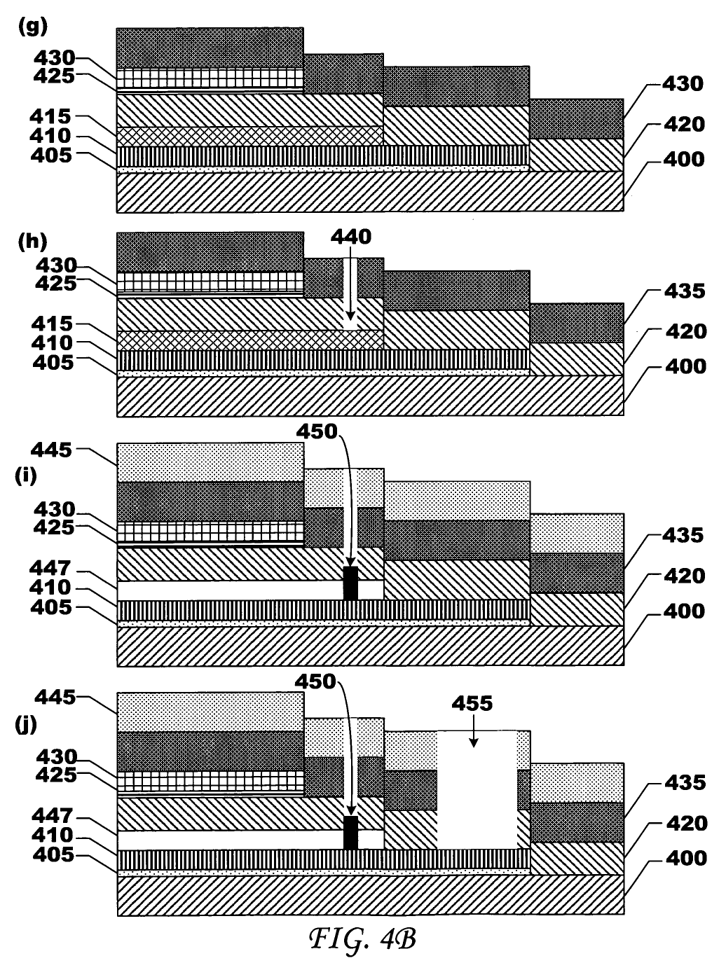

FIGS. 4A and 4B (collectively FIG. 4) illustrate another preferred fabrication process utilized to produce a cMUT on a substrate in accordance with the present invention. Specifically, FIGS. 4(a)-(j) (FIGS. 4(a)-4(f) are shown in FIG. 4A, and 4(g)-4(j) are shown in FIG. 4B) illustrate a cMUT fabrication process that requires only five masks, reduces processing time over conventional processes, utilizes etch resistant metals as conductive layers to form cMUT electrodes, and does not utilize an isolation layer. The fabrication process illustrated in FIGS. 4(a)-(j) is described with specific metal layers and specific layer thicknesses, although the invention can be implemented with other metals and different layer thicknesses. In addition, it will be understood that alternative conductive materials can be used in the place of the metals disclosed. Further, the fabrication process illustrated in FIGS. 4(a)-(j) can be performed in various orders.

In a first step, multiple metal layers can be applied to a substrate 400. For example, a first metal layer 405 of Chromium can be applied onto the substrate 400, and can have a thickness of approximately 200 Angstroms. The first metal layer 405 can be an adhesion layer ensuring that any layer placed on the first metal layer 405 adequately adheres to the substrate 400. An adhesion layer is not necessary of subsequent layers adequately adhere to the substrate 400.

A second metal layer 410 is then deposited onto the first metal layer 405. The second metal layer 410 can be Gold, and can have a thickness of approximately 1000 to approximately 1500 Angstroms. The second metal layer 410 can form a first, or ground, electrode for a cMUT device. Next, a third metal layer 415 can be deposited onto the second metal layer 410.

A third metal layer 415 can be Chromium, and preferably has a thickness of approximately 1000 to approximately 1500 Angstroms. The third metal layer 415 can be a sacrificial layer in some embodiments. The combination of Gold and Chromium for a bottom electrode and a sacrificial layer is advantageous because etchants are readily available that will etch Chromium while leaving Gold unaffected. For example, Chromium Etchant CRE-473 from Transene Company, Inc. may be used as the etchant. Alternatively, this advantage may be realized by a combination of bottom electrode (second metal layer 410) and sacrificial layer materials exhibiting this same etchant relationship. Accordingly, it will be appreciated that Gold and Chromium are provided as examples of suitable materials for the present invention and alternative materials can be used.

Additionally, it is desirable to use a bottom electrode that is not affected by an etchant that is used with the sacrificial layer because it eliminates the need for an isolation layer. The isolation layer, while protecting the bottom electrode from etchants, contributes to parasitic capacitance. The isolation layer can also reduce the efficiency of the cMUT and cause charging problems. Eliminating the isolation layer can reduce such parasitic capacitance, increase cMUT efficiency, and eliminate potential charging problems.

After the first, second, and third metal layers 405, 410, and 415 have been deposited onto the substrate 400, they can be patterned if desired for a particular application, or alternatively can be patterned during individual deposition. For example, as shown in FIG. 4(b), the third metal layer 415 can be patterned to have a different geometrical configuration than the substrate 400. Additionally, as illustrated in FIG. 4(c), the first and second metal layers 405, 410 can also be patterned to have different geometrical configurations than the substrate 400. In some embodiments, the first and second metal layers 405, 410 can be patterned similarly, and in other embodiments, they may be patterned differently. The first, second, and third metal layers 405, 410, 415 may be patterned using a wet etch, and cleaned in an ultrasonic cleaner with a cold Acetone bath.

In a next step, a first membrane layer 420 is deposited onto the first, second, and third metal layers 405, 410, 415 and the substrate 400 as illustrated in FIG. 4(d). The first membrane layer 420 can be a layer of silicon nitride, preferably having a thickness of approximately 6000 Angstroms. The first membrane layer 420 can be deposited using a Unaxis 790 PECVD system. After the first membrane layer 420 is deposited, additional metal layers can be deposited on the first membrane layer 420.

The metal layers deposited onto the first membrane layer 420 can include an adhesive layer and a layer forming the top electrode for a cMUT. For example, a fourth metal layer 425 can be a layer of Chromium, preferably having a thickness of approximately 200 Angstroms. The fourth metal layer 425 can be an adhesion layer ensuring that any layer placed onto the fourth metal layer 425 adequately adheres to the first membrane layer 420.

A fifth metal layer 430 can be deposited on the fourth metal layer 425 as illustrated in FIG. 4(e). The fifth metal layer 430 can be Gold, preferably having a thickness of approximately 1000 to approximately 1500 Angstroms. The fifth metal layer 430 can be patterned to form a top electrode for a cMUT. Such patterning is illustrated in FIG. 4(f), wherein, the fourth and fifth metal layers 425, 430 are patterned to have a different geometrical configuration than the substrate 400. In some embodiments, the fourth and fifth metal layers 425, 430 may be patterned using a wet etch, and cleaned in an ultrasonic cleaner with a cold Acetone bath.

In a next step, a second membrane layer 435 is deposited onto the fourth an fifth metal layers 425, 430 and the first membrane layer 420, as illustrated in FIG. 4(g). The second membrane layer 435 can be a layer of silicon nitride, preferably having a thickness of approximately 6000 Angstroms. The second membrane layer 435 can be deposited using a Unaxis 790 PECVD system. After the second membrane layer 435 is deposited, the first and second membrane layers can be patterned to form a release aperture or hole 440. Although only a single release hole 440 is illustrated in FIG. 4(h), the present invention can utilize multiple release holes 440. Once the release hole 440 is formed, the third metal layer 415 can be etched or removed using a RIE or wet etching process. The bottom electrode (second metal layer 410) can be a material resistant to an etchant capable of etching a sacrificial layer and not damaging the bottom electrode (second metal layer 410).

Removing or etching the third metal layer 415 can form a cavity 447 as illustrated in FIG. 4(i). The cavity 447 can be disposed between the first and second metal layers 405, 410, and the fourth and fifth metal layers 425, 430. The first membrane layer can define the cavity 447. The cavity 447 can be sealed with a seal 450 and by deposition of a third membrane layer 445. The cavity 447 enables fluctuation of the cMUT membrane formed by the first, second, and third membrane layers 420, 435, 445, and resonance in response to stimuli.

The third membrane layer 445 can be deposited onto the second membrane layer 435. The third membrane layer 445 can be a sealing layer, and preferably has a thickness of approximately 6000 Angstroms. The third membrane layer 445 can be deposited using a Unaxis 790 PECVD system. The third membrane layer 445 can be patterned to have a predetermined geometric configuration so the combined thickness of the second and third membrane layers 435, 445 is a predetermined thickness. As illustrated in FIG. 4(j), the membrane formed by the first, second, and third membrane layers 420, 435, 445 can suspend the fourth and fifth metal layers 425, 430 above the cavity 447.

In a last step, the first, second, and third membrane layers 420, 435, 445 can be patterned to form a connection area 455. The connection area 455 can be adapted for bond pads enabling connections to be made to the second metal layer 410. Similarly, although not shown, a connection area can be formed to provide access to the fourth metal layer 425. The first, second, and third membrane layers 420, 435, 445 can be etched using an RIE or wet etching processes. After the connection area 455 is formed, the resulting fabricated device can be cleaned in an ultrasonic cleaner with a cold Acetone bath.

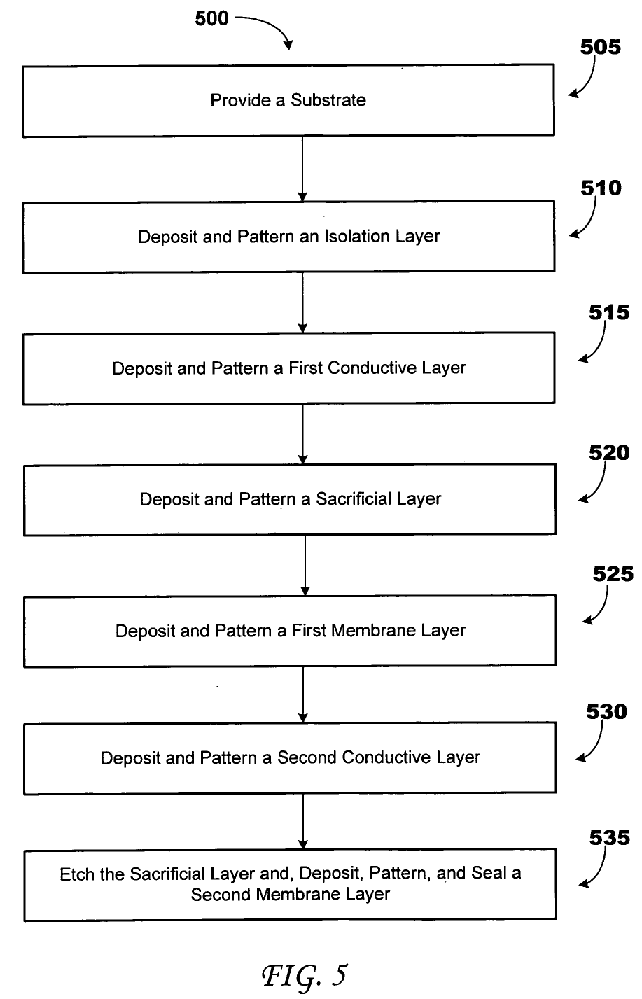

FIG. 5 is a logic flow diagram depicting a method of fabricating a cMUT device. The first step involves providing a substrate (step 505), the substrate preferably being an opaque or transparent substrate. Next, an isolation layer can be deposited onto the substrate, and patterned to have a predetermined thickness (step 510). After the isolation layer is patterned, a first conductive layer can be deposited onto the isolation layer, and patterned into a predetermined configuration (step 515). The first conductive layer can form a bottom electrode for a cMUT on a substrate. Once the first conductive layer is patterned into a predetermined configuration, a sacrificial layer can be deposited onto the first conductive layer (step 520). The sacrificial layer can be patterned by selective deposition and patterning techniques so that it has a predetermined thickness. Then, a first membrane layer can be deposited onto the sacrificial layer (step 525).

The deposited first membrane layer is then patterned to have a predetermined thickness, and a second conductive layer is then deposited onto the first membrane layer (step 530). The second conductive layer can form a top electrode for a cMUT. After the second conductive layer is patterned into a predetermined configuration, a second membrane layer can be deposited onto the patterned second conductive layer (step 535). The second membrane layer can also be patterned to have an optimal geometric configuration.

The first and second membrane layers can encapsulate the second conductive layer, enabling it to move relative to the first conductive layer due to elastic characteristics of the first and second membrane layers. After the second membrane layer is patterned, the sacrificial layer can be etched away, forming a cavity between the first and second conductive layers (step 535). The cavity formed below the first and second membrane layers can provide space for the resonating first and second membrane layers to move relative to the substrate. In a last part of this step, the second membrane layer can be sealed by depositing a sealing layer onto the second membrane layer (step 535).

The various embodiments of the present invention can also be utilized to form an array of cMUTs for a cMUT imaging system. Those skilled in the art will recognize that the cMUT imaging arrays illustrated in FIGS. 6 and 7 are only exemplary, and that other imaging arrays are achievable in accordance with the embodiments of the present invention.

FIG. 6 illustrates a cMUT imaging array device formed in a ring-annular array on a substrate. As shown, the device 600 includes a substrate 605 and cMUT arrays 610, 615. The substrate 605 is preferably disc-shaped, and the device 600 may be utilized as a forward looking cMUT imaging array. Although the device 600 is illustrated with two cMUT arrays 610, 615, other embodiments can have one or more cMUT arrays. If one cMUT array is utilized, it can be placed near the outer periphery of the substrate 605. If multiple cMUT arrays are utilized, they can be formed concentrically so that the circular-shaped cMUT arrays have a common center point. Some embodiments can also utilize cMUT arrays having different geometrical configurations in accordance with some embodiments of the present invention.

FIG. 7 illustrates a cMUT imaging array system formed in a side-looking array on a substrate. As shown, the device 700 includes a substrate 705, and cMUT arrays 710, 715. The substrate 705 can be cylindrically-shaped, and the cMUT arrays can be coupled to the outer surface of the substrate 705. The cMUT arrays 710, 715 can comprise cMUT devices arranged in an interdigital fashion and used for a side-looking cMUT imaging array. Some embodiments of device 700 can include one or multiple cMUT imaging arrays 710, 715 in spaced apart relation on the outer surface of the cylindrically-shaped substrate 700.

While the various embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all applicable equivalents.

1. A method of fabricating a cMUT on a substrate having a surface at a process temperature, the method comprising:
providing a first conductive layer proximate the surface of the substrate, the first conductive layer being resistant to an etchant;
providing a sacrificial layer proximate a portion of the first conductive layer; and
etching the cMUT with the etchant, wherein the etchant etches a portion of the sacrificial layer.

2. The method of claim 1, further comprising:
providing a first membrane layer proximate the sacrificial layer;
providing a second conductive layer proximate a portion of the first membrane layer; and
providing a second membrane layer proximate the second conductive layer.

3. The method of claim 1, wherein the process temperature is less than approximately 300 degrees Celsius.

4. The method of claim 1, wherein the substrate comprises an embedded circuit.

5. The method of claim 1, wherein the first conductive layer comprises Gold.

6. The method of claim 1, wherein the sacrificial layer comprises Chromium.

7. The method of claim 1, further comprising providing a transparent substrate as the substrate.

8. The method of claim 1, further comprising providing a reflective layer as at least one of the first conductive layer, the second conductive layer, the first membrane layer, and the second membrane layer.

9. The method of claim 1, further comprising providing a circuit proximate the substrate adapted to receive and provide optical signals.

10. A cMUT device comprising:
a first conductive layer of the cMUT device proximate a substrate, the first conductive layer being resistant to an etchant; and
a first membrane layer of the cMUT proximate the first conductive layer, the first membrane layer defining a cavity formed by etching a sacrificial layer with the etchant.

11. The device of claim 10 further comprising:
a second conductive layer proximate the first membrane layer; and
a second membrane layer proximate the second conductive layer.

12. The device of claim 10, further comprising a circuit proximate the substrate to direct and receive and at least one of an optical and electrical signal to and from the first conductive layer.

13. The device of claim 10, wherein the substrate enables at least one of an electrical or optical signal to pass through the substrate.

14. The device of claim 10, wherein the first conductive layer comprises Gold and the sacrificial layer comprises Chromium.

15. The device of clam 10, wherein at least one of the first conductive layer is placed proximate the substrate at a temperature of less than approximately 300 degrees Celsius.

16. The device of claim 10, wherein the substrate comprises an embedded circuit.

17. A method of fabricating a cMUT on a substrate having a surface, the method consisting of:
providing a first conductive layer proximate the surface of the substrate, the first conductive layer being resistant to an etchant;
providing a sacrificial layer proximate at least a portion of the first conductive layer;
providing a first membrane layer proximate the sacrificial layer;
providing a second conductive layer proximate at least a portion of the first membrane layer;
providing a second membrane layer proximate the second conductive layer; and
removing at least a portion of the sacrificial layer with the etchant.

18. The method of claim 17, further consisting of disposing an adhesion layer between the surface of the substrate and first conductive layer.

19. The method of claim 17, further comprising at least one of the first conductive layer, the second conductive layer, and the sacrificial layer at a temperature of less than 300 degrees Celsius.

20. The method of claim 17, wherein the substrate is adapted to enable at least one of an optical or electrical signal to pass through the substrate.
